(19) United States
(12) Patent Application Publication
(10) Pub. No.: US 2023/0124369 A1
(43) Pub. Date: Apr. 20, 2023

(54) HEALTH DETERMINATIONS FROM TRACHEAL SOUND AND ORAL EXPIRATORY FLOW

(71) Applicant: HEWLETT-PACKARD DEVELOPMENT COMPANY, L.P., Spring, TX (US)

(72) Inventors: Hsiang-Ta Ke, Taipei (TW); Shih-Hsin Chen, Taipei (TW); Chih-Cheng Chan, Taipei (TW)

(73) Assignee: HEWLETT-PACKARD DEVELOPMENT COMPANY, L.P., Spring, TX (US)

(21) Appl. No.: 17/502,671

(22) Filed: Oct. 15, 2021

(51) Int. Cl. A61B 5/08; G10L 25/66; H04R 1/08; H04R 1/02; A61B 7/00; A61B 5/00; A61B 5/20

(52) U.S. Cl. CPC A61B 5/082; G10L 25/66; H04R 1/08; H04R 1/02; A61B 7/003; A61B 5/4244; A61B 5/201; A61B 5/6803

(57) ABSTRACT

In one example in accordance with the present disclosure, an electronic device is described. The example electronic device includes a microphone device to record a tracheal sound. The example electronic device also includes an oral expiratory flow sensor to measure oral expiratory flow contents. The example electronic device further includes a processor to determine a health condition based on the tracheal sound and the oral expiratory flow contents.

### BACKGROUND

Electronic technology has advanced to become virtually ubiquitous in society and has been used to enhance many activities in society. For example, electronic devices are used to perform a variety of tasks, including work activities, communication, research, and entertainment. Different varieties of electronic circuits may be utilized to provide different varieties of electronic technology.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of an electronic device to determine health conditions, according to an example.

FIG. 2 illustrates a headset to determine health conditions, according to an example.

FIG. 3 is a block diagram illustrating an oral expiratory flow sensor, according to an example.

FIG. 4 is a flow diagram illustrating a method for determining a health condition, according to an example.

FIG. 5 depicts a non-transitory machine-readable storage medium for determining a health condition, according to an example.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Electronic devices are used by millions of people daily to carry out business, personal, and social operations and it is not uncommon for an individual to interact with multiple electronic devices on a daily basis. Examples of electronic devices include desktop computers, laptop computers, all-in-one devices, tablets, medical devices, and gaming systems to name a few. Other examples of electronic devices include sensors that interact with users.

An electronic device may include any number of hardware components. These hardware components operate with other hardware components to execute a function of the electronic device. For example, a memory device may include instructions that are executable by a processor. The instructions when executed by the processor, may cause the processor to execute an operation on the electronic device. As a specific example, the electronic device may include a central processing unit (CPU) and/or a graphics processing unit (GPU). The electronic device also includes circuitry to interconnect the hardware components. For example, the processor of an electronic device may perform operations based on data received from hardware components. For example, sensors may provide signals to a processor, which then performs an operation based on the signals. While specific reference is made to particular hardware components, an electronic device may include any number and any variety of hardware components to carry out an intended function of the electronic device.

As electronic devices are becoming more ubiquitous in society, some developments may further enhance their integration. For example, a user may interact with the electronic device in such a manner that the electronic device can measure physical characteristics of the user. These measurements may be used by an electronic device to determine health conditions of the user.

As a particular example, some electronic devices may include sensors to measure the contents of a user's breath as the user exhales through their mouth. These sensors are referred to herein as oral expiratory flow sensors. In some examples, electronic devices may include a microphone to capture sounds produced by a user. For example, the microphone may record tracheal sounds produced by the user. Electronic devices may make determinations about the health condition of the user based on these measurements of the user.

The present specification describes examples of an electronic device. The electronic device includes a microphone device to record a tracheal sound. The electronic device also includes an oral expiratory flow sensor to measure oral expiratory flow contents. The electronic device further includes a processor to determine a health condition based on the tracheal sound and the oral expiratory flow contents.

In another example, the present specification also describes a method that includes receiving, at a processor, a measurement of oral expiratory flow contents. The method also includes determining, by the processor, a health condition based on the oral expiratory flow contents.

In yet another example, the present specification also describes a non-transitory computer-readable storage medium comprising instructions executable by a processor to receive a tracheal sound recording from a microphone device. The instructions are also executable by the processor to receive a measurement of oral expiratory flow contents from an oral expiratory flow sensor. The instructions are further executable by the processor to determine a lung condition based on the tracheal sound recording and the oral expiratory flow contents.

As used in the present specification and in the appended claims, the term “processor” may be a processor resource, a controller, an application-specific integrated circuit (ASIC), a semiconductor-based microprocessor, a central processing unit (CPU), and a field-programmable gate array (FPGA), and/or other hardware device that executes instructions.

As used in the present specification and in the appended claims, the term “memory” may include a computer-readable storage medium, where the computer-readable storage medium may contain, or store computer-usable program code for use by or in connection with an instruction execution system, apparatus, or device. The memory may take many types of memory including volatile memory (e.g., RAM) and non-volatile memory (e.g., ROM).

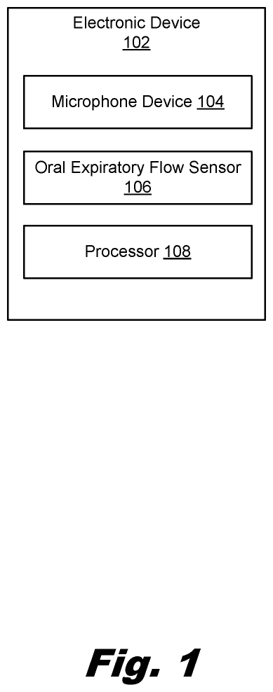

Turning now to the figures, FIG. 1 is a block diagram of an electronic device 102 to determine health conditions, according to an example. Examples of electronic devices include compute devices, workstations, servers, laptop computers, desktop computers, smartphones, tablet devices, wireless communication devices, sensors, headsets, wireless earbuds, etc.

The electronic device 102 may include memory resources and processing resources to perform computing tasks. For example, memory resources may include volatile memory (e.g., random-access memory (RAM)) and non-volatile memory (e.g., read-only memory (ROM), data storage devices (e.g., hard drives, solid-state devices (SSDs), etc.) to store data and instructions. In some examples, processing resources may include circuitry to execute instructions. Examples of processing resources include a central processing unit (CPU), a graphics processing unit (GPU), or other hardware device that executes instructions, such as an application specific integrated circuit (ASIC).

In some examples, the electronic device 102 may include a microphone device 104 to record a tracheal sound. In some examples, the microphone device 104 may include a transducer to convert sound waves into an electrical signal. In some examples, the microphone device 104 may output a recording of detected sounds.

In some examples, the microphone device 104 may include a single microphone. In some examples, the microphone device 104 may include an array of microphones where a plurality of microphones are used to obtain a sound recording. In some examples, the microphone array may provide a high-definition recording.

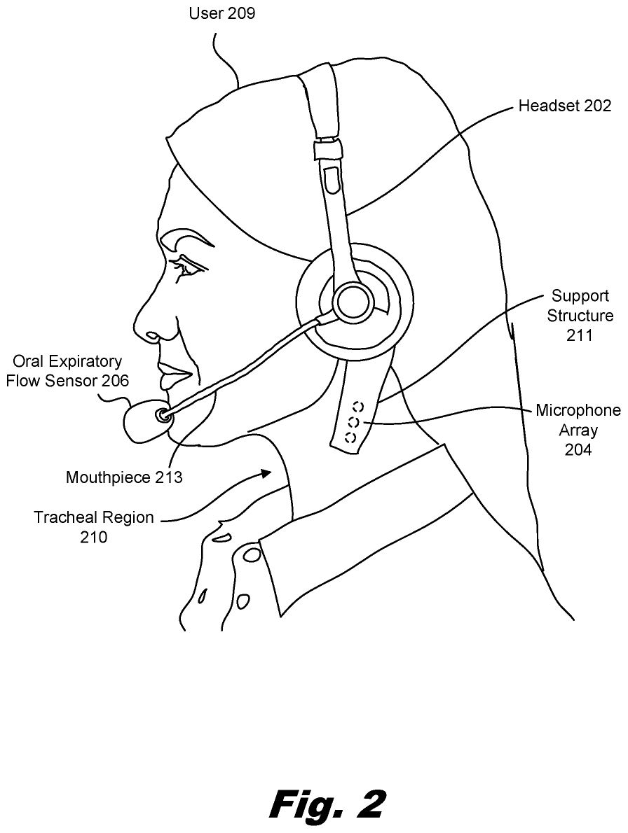

In some examples, the microphone device 104 may be positioned on a support structure (not shown) attached to the electronic device 102. The support structure may facilitate locating the microphone device 104 in proximity to a tracheal region of a human user. In humans, the trachea (also referred to as the windpipe) is a tube that connects the larynx to the lungs, allowing for the passage of air. The support structure may position the microphone device 104 in proximity to the tracheal region of a user such that the microphone device 104 is oriented to capture sounds emanating from the trachea of the user. In some examples, the microphone of the microphone device 104 may contact the skin of the throat of a user to obtain the tracheal sounds of the user. An example of the microphone device 104 included in a headset is described in FIG. 2.

In some examples, the microphone device 104 may output a sound recording. For example, the microphone device 104 may output a compressed audio file format or an uncompressed audio file format. Examples of sound recording formats include Waveform (WAV) files, MP3, AAC, Vorbis, FLAC, etc. In some examples, the microphone device 104 may output electrical signals to a processor 108, which may then process the electrical signals to generate sound recordings.

The electronic device 102 may include an oral expiratory flow sensor 106 to measure oral expiratory flow contents. In some examples, the oral expiratory flow sensor 106 may be a device that measures the contents of a user's breath as the user exhales. The oral expiratory flow sensor 106 may be positioned on the electronic device 102 to encounter the exhaled breath (referred to herein as the oral expiratory flow) of a user.

In some examples, the oral expiratory flow sensor 106 may vibrate based on adhesion of the oral expiratory flow contents on the oral expiratory flow sensor 106. For example, the oral expiratory flow sensor 106 may include an oscillator (e.g., piezoelectric oscillator, quartz oscillator, etc.) that vibrates. As a user exhales, chemical substances in the oral expiratory flow may temporarily adhere to the oscillator. The vibration frequency of the oscillator changes based on changes in the oral expiratory flow contents adhered to the oscillator. For example, one chemical substance has a given weight (or mass) per unit area while a different chemical substance has a different weight (or mass) per unit area. Thus, the presence of different chemical substances on the oral expiratory flow sensor 106 may cause the oral expiratory flow sensor 106 to vibrate with different frequencies.

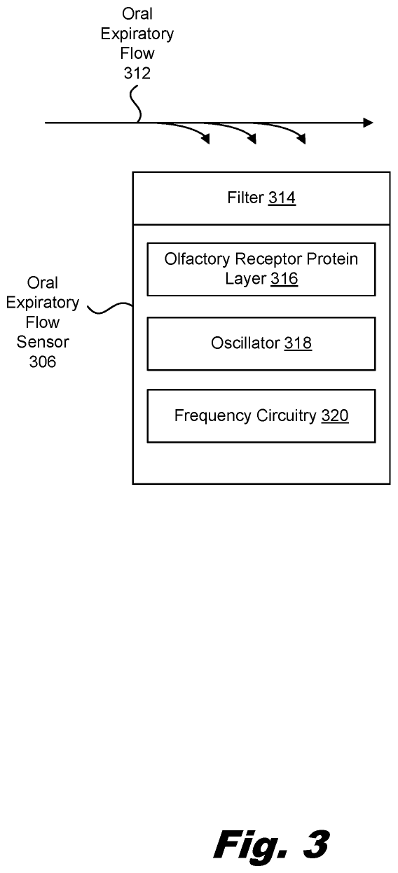

The contents of the oral expiratory flow may be determined based on the difference in a measured oscillator frequency to a baseline frequency where no oral expiratory flow contents are present on the oral expiratory flow sensor 106. The frequency of the oscillator indicates that a type of chemical substance is present in the oral expiratory flow. An example of this determination is described in FIG. 3.

In some examples, the oral expiratory flow sensor 106 may output the frequency of the oscillator. For example, the oral expiratory flow sensor 106 may output the frequency of the oscillator to the processor 108, which then determines the oral expiratory flow contents based on the frequency measurement. In some examples, the oral expiratory flow sensor 106 may determine the oral expiratory flow contents, which are then communicated to the processor 108.

As mentioned above, the electronic device 102 includes a processor 108. The processor 108 of the electronic device 102 may be implemented as dedicated hardware circuitry or a virtualized logical processor. In some examples, the dedicated hardware circuitry may be implemented as a central processing unit (CPU). A dedicated hardware CPU may be implemented as a single to many-core general purpose processor. A dedicated hardware CPU may also be implemented as a multi-chip solution, where more than one CPU are linked through a bus and schedule processing tasks across the more than one CPU. In some examples, the processor 108 may be implemented as an embedded controller with firmware.

In some examples, memory may be implemented in the electronic device 102. The memory may be dedicated hardware circuitry to host instructions for the processor 108 to execute. In another implementation, the memory may be virtualized logical memory. Analogous to the processor 108, dedicated hardware circuitry may be implemented with dynamic random-access memory (DRAM) or other hardware implementations for storing processor instructions. Additionally, the virtualized logical memory may be implemented in an abstraction layer which allows the instructions to be executed on a virtualized logical processor, independent of any dedicated hardware implementation.

The electronic device 102 may also include instructions. The instructions may be implemented in a platform specific language that the processor 108 may decode and execute. The instructions may be stored in the memory during execution. In some examples, the instructions may cause the processor 108 to determine a health condition based on the tracheal sound and the oral expiratory flow contents, according to the examples described herein.

In some examples, the health condition determination may be based on whether the amounts of chemical substances measured in the oral expiratory flow is greater than threshold amounts. In addition to containing nitrogen, oxygen, and carbon dioxide, oral expiratory flow may contain more than 400 kinds of detectable chemical substances (e.g., molecules, volatile organic compounds, etc.). Oral expiratory flow may indicate a human's health condition. For example, a person with kidney failure may experience abnormal concentrations of ammonia, dimethylamine, trimethylamine, or a combination thereof. A person with liver disease may have abnormal concentrations of ammonia, trimethylamine, acetic acid and mercaptans. A person with diabetes may have abnormal concentrations of acetone. Therefore, the measured oral expiratory flow contents may be used to determine health conditions of the user of the electronic device 102.

The oral expiratory flow sensor 106 may measure certain chemical substances in the oral expiratory flow of a user. The amount of the chemical substances may be determined either by the oral expiratory flow sensor 106 or the processor 108. The processor 108 may compare the measured oral expiratory flow contents to thresholds to determine whether a health condition is detected.

The following examples illustrate different chemical substances and thresholds that may be measured to determine health conditions. In some examples, the oral expiratory flow sensor 106 may measure volatile sulfur and hydrogen sulfide to determine a mouth condition (e.g., bad breath). In this example, the mouth condition may be detected if volatile sulfur is greater than 5 parts-per-million (ppm) and hydrogen sulfide is greater than 5 ppm.

In another example, the processor 108 may detect a diabetes condition based on the measured amount of acetone in the oral expiratory flow. For example, a diabetes condition may be detected if the measured acetone amount is greater than 5 ppm and less than 300 ppm (i.e., 5 ppm<Acetone<300 ppm).

In another example, the processor 108 may detect a liver condition based on the measured amount of ammonia and acetic acid in the oral expiratory flow. For example, a liver condition may be detected if the ammonia measurement is greater than 1 ppm, while the acetic acid measurement is greater than 1 ppm and less than 7 ppm (i.e., ammonia>1 ppm; 1 ppm<acetic acid<7 ppm).

In another example, the processor 108 may detect a kidney condition based on the measured amount of ammonia in the oral expiratory flow. For example, a kidney condition may be detected if the ammonia measurement is greater than 4.8 ppm (i.e., ammonia>4.8 ppm).

In some examples, the health condition may be determined based on a combination of the tracheal sound provided by the microphone device 104 and the oral expiratory flow measurement provided by the oral expiratory flow sensor 106. For example, the processor 108 may receive a tracheal sound recording from the microphone device 104. The processor 108 may also receive a measurement of oral expiratory flow contents from the oral expiratory flow sensor 106. The processor may then determine a lung condition based on the tracheal sound recording and the oral expiratory flow contents.

In some examples, to determine the lung condition, the processor 108 may determine whether a trimethylamine measurement in the oral expiratory flow is greater than a threshold (e.g., greater than 5 ppm). If the trimethylamine measurement in the oral expiratory flow is greater than the threshold, then the processor 108 may determine whether the tracheal sound recording matches a sound signature for tracheitis. As used herein, tracheitis refers to inflammation of the trachea. Tracheitis may be caused by an infection in the respiratory tract.

In some examples, tracheitis may include different conditions that each have different sound signatures. Different forms of tracheitis include wheezing, crackles, and stridor. In the case of wheezing, this lung condition is characterized by a high pitch sound. The sound may be made by an upper airway obstruction, which can be heard during the inhalation period. The sound signature for wheezing is a frequency of approximately 100 hertz (Hz) (e.g., 80 Hz<frequency<120 Hz) in a 100 millisecond (ms) duration.

In the case of crackles (also referred to as croup), this lung condition is a symptom of an upper airway obstruction disease. The sound signature for crackles is a frequency around 200-2000 Hz (e.g., 200 Hz<frequency<2000 Hz) and a duration of less than 20 ms.

In the case of stridor, this lung condition is characterized by a whistling sound with a high pitch that can be heard when breathing. The sound signature for stridor is a frequency of approximately 400 HZ (e.g., 350 Hz<frequency<420 Hz) and a duration greater than 250 ms.

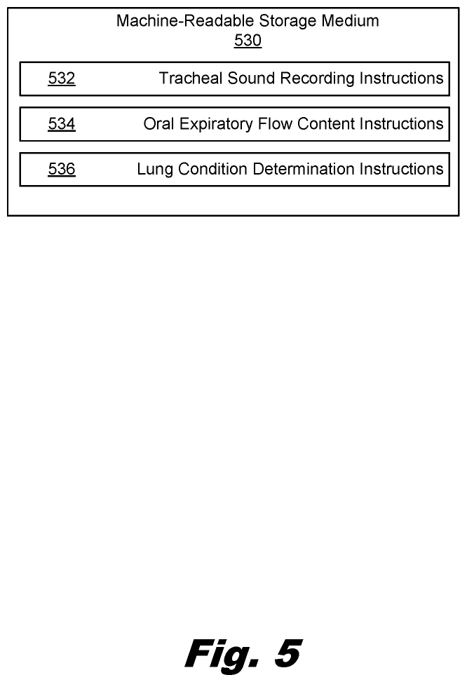

The processor 108 may analyze samples (e.g., portions) of the tracheal sound recording provided by the microphone device 104 to determine whether the tracheal sound recording matches a sound signature for tracheitis. It should be noted that the processor 108 may determine that one, or multiple lung conditions are present if the tracheal sound recording matches a single or multiple sound signatures for tracheitis. Examples of determining a lung condition based on the tracheal sound are described further in FIG. 5.

In some examples, the processor 108 may generate an alert or notification in the event that a health condition is identified. In some examples, the alert may be in the form of a sound or recording notifying the user of the health condition. In the example that the electronic device 102 is a headset, the processor 108 may cause an audible message to play informing the user of the health condition. For example, if a mouth condition is identified, then the processor 108 may cause an audible message to play in the headset stating, “Mouth condition detected.” It should be noted that in other examples, different messages may be generated for given health conditions.

In some examples, the processor 108 may send a signal to a remote compute device (e.g., a desktop computer, laptop computer, smartphone, etc.) to generate an alert or notification in the event that a health condition is identified. For example, the electronic device 102 may include network communication circuitry for communicating with a remote compute device. The processor 108 may send a health condition signal to the remote compute device using the network communication circuitry. In some examples, a remote compute device may be referred to as a peripheral device.

In some examples, the remote compute device may include a host daemon to monitor for health condition signals from the processor 108. Upon receiving a health condition signal for a given health condition, the host daemon may cause a notification (e.g., audio or video notification) to be generated. In some examples, the host daemon may cause a warning message to be displayed on a display device (e.g., a monitor) that indicates the detection of the health condition. In some examples, the host daemon may send a message (e.g., an email, text message, etc.) to a given account with details about the health condition in response to receiving the health condition signal from the processor 108.

In some examples, the electronic device 102 may perform an action in response to measurements of the tracheal sound, the oral expiratory flow contents, or a combination of both. For example, the electronic device 102 hosting the microphone device 104 and oral expiratory flow sensor 106 may change a configuration based on the tracheal sound measurements and/or oral expiratory flow measurements. In some examples, the electronic device 102 may increase measurement sampling rates of the tracheal sounds and oral expiratory flow upon detection of a health condition. Other examples of configuration changes by the electronic device 102 may include communicating the health condition to a peripheral device based on the tracheal sound measurements and/or oral expiratory flow measurements. In some examples, the electronic device 102 may include communication circuitry (not shown) that is activated in response to the tracheal sound measurements and/or oral expiratory flow measurements. The communication circuitry may be used to activate a peripheral device to perform an action based on the tracheal sound measurements and/or oral expiratory flow measurements.

In some examples, the electronic device 102 may include components (not shown) to perform a medical procedure based on the tracheal sound measurements and/or oral expiratory flow measurements. For example, the electronic device 102 may include an oxygen delivery device that is activated or deactivated based on the tracheal sound measurements and/or oral expiratory flow measurements to provide oxygen to the user.

In some examples, a peripheral device may perform an action in response to measurements of the tracheal sound, the oral expiratory flow contents, or a combination of both. For example, the electronic device 102 may send a signal to a peripheral device based on the tracheal sound measurements, the oral expiratory flow measurements, or both. The peripheral device may perform an action based on the tracheal sound measurements or the oral expiratory flow measurements. In some examples, the peripheral device changes a configuration (e.g., a setting, parameter, etc.) based on the tracheal sound measurements or the oral expiratory flow measurements. Some examples of configurations include power settings, display settings, network communication settings, etc.

In some examples, the peripheral device may perform a medical process based on the tracheal sound measurements or the oral expiratory flow measurements. For example, the peripheral device may perform a dialysis process based on the oral expiratory flow measurements indicating a kidney health condition. In another example, the peripheral device may perform an insulin delivery process based on the oral expiratory flow measurements indicating a diabetes health condition. In some examples, the peripheral device may perform a pulmonary therapy process (e.g., adjust administered oxygen levels) based on tracheal sound measurements and the oral expiratory flow measurements.

In some examples, the electronic device 102 or a peripheral device may monitor the health conditions detected by the electronic device 102. For example, the electronic device 102 or the peripheral device may log the tracheal sound measurements or the oral expiratory flow measurements to generate a health history of the user. In some examples, the electronic device 102 or the peripheral device may perform an action based on the tracheal sound measurement and oral expiratory flow measurement history.

FIG. 2 illustrates a headset 202 to determine health conditions, according to an example. In this example, the headset 202 may be implemented according to the electronic device 102 of FIG. 1.

The headset 202 may be worn on the head of a user 209. For example, the headset 202 may include an oral expiratory flow sensor 206 and a microphone array 204. In this example, the oral expiratory flow sensor 206 may be located at the end of a mouthpiece 213 such that the oral expiratory flow sensor 206 may be positioned in the oral expiratory flow of the user 209. Examples of various structures for the oral expiratory flow sensor 206 are described in FIG. 3.

The headset 202 may include a microphone array 204 coupled to a support structure 211. In some examples, the support structure 211 may position the microphone array 204 in proximity to a tracheal region 210 of the human user 209. In some examples, the support structure 211 may couple to an earpiece of the headset 202. In some examples, the support structure 211 may provide for positioning of the microphone array near the tracheal region 210. In some examples, the support structure 211 may allow the microphone array 204 to contact the skin of the user 209 in the tracheal region 210.

In some examples, the microphone array 204 may include a single microphone. In some examples, the microphone array 204 may include multiple microphones.

In some examples, the headset 202 may include a processor (not shown) to determine a health condition based on the tracheal sound and the oral expiratory flow contents. For example, the oral expiratory flow sensor 206 may provide a measurement of oral expiratory flow contents to the processor. The microphone array 204 may provide a tracheal sound recording to the processor. In some examples, the measurements of oral expiratory flow contents and tracheal sound recording may be provided on a continuous basis or on a periodic basis. Upon receiving the measurements of oral expiratory flow contents or tracheal sound recording, the processor may determine whether the user 209 is experiencing a health condition.

FIG. 3 is a block diagram illustrating an oral expiratory flow sensor 306, according to an example. The oral expiratory flow sensor 306 may act as an electro-chemical gas sensor. In this example, the oral expiratory flow sensor 306 includes a filter 314. The filter 314 may encounter the oral expiratory flow 312 as a person exhales. The filter 314 may block large particles while allowing smaller chemical substances to pass through openings in the filter 314. In some examples, the filter 314 may be a mechanical filter made of fiber (e.g., glass fiber, paper fiber, polymer fiber, etc.). The filter 314 may use multiple layers of fiber to catch particles in the oral expiratory flow 312. For example, the filter 314 may catch saliva, dust, or other large particles through inertial impact.

The oral expiratory flow sensor 306 may include an olfactory receptor protein layer 316 coupled to the surface of an oscillator 318. The olfactory receptor protein layer 316 may receive filtered oral expiratory flow contents. The filtered contents of the oral expiratory flow 312 may include chemical substances. The olfactory receptor protein layer 316 may include proteins that bond to certain chemical substances. In some examples, the olfactory receptor protein layer 316 may be formed from G-protein-coupled receptors to coat the surface of a multi-element array of piezoelectric crystals.

In some examples, a given protein may be used to form an olfactory receptor protein layer 316 that adheres to a given chemical substance. For example, a first protein may be used to adhere to a first chemical substance (e.g., volatile sulfur), a second protein may be used to adhere to a second chemical substance (e.g., hydrogen sulfide), and so forth.

The mass of the oscillator 318 will change as chemical substances from the oral expiratory flow 312 adhere to the olfactory receptor protein layer 316. Because the olfactory receptor protein layer 316 is coupled to the surface of an oscillator 318, adhesion of a chemical substance on the olfactory receptor protein layer 316 may change the vibration frequency of the oscillator 318. Thus, the oscillator 318 may vibrate with a frequency based on adhesion of the oral expiratory flow contents on the olfactory receptor protein layer 316. Different adhesion molecules and compounds may result in different weight changes, so the type of the adhesion molecules can be calculated by the frequency of the oscillator 318. Equation (1) may be used to determine the mass of the chemical substance adhered to the olfactory receptor protein layer 316.

$${F}_{2} - {F}_{1} = C * {F}_{1} * {F}_{1} * ( \frac{( {M}_{2} - {M}_{1 )}}{A} ) . \qquad ( 1 )$$

In Equation (1), F₁ is a baseline frequency of the oscillator 318 without any chemical substance adhered to the olfactory receptor protein layer 316. F₂ is the frequency of the oscillator 318 with a chemical substance adhered to the olfactory receptor protein layer 316, where F₂ changes based on the oral expiratory flow 312. C is a constant. M₁ is the mass of the oscillator and olfactory receptor protein layer 316 without any chemical substance adhered to the olfactory receptor protein layer 316. M₂ is the mass of the oscillator, olfactory receptor protein layer 316 and the chemical substance adhered to the olfactory receptor protein layer 316. A is the area of contact surface of the olfactory receptor protein layer 316. By determining the difference in frequencies (F₂−F₁), the mass of the chemical substance adhered to the olfactory receptor protein layer 316 may be determined according to Equation (1).

In some examples, a given protein in the olfactory receptor protein layer 316 may be selected to adhere to a given chemical substance. Thus changes in the frequency of the oscillator 318 may indicate an amount of a given chemical substance.

In some examples, multiple oscillators 318 may be used to measure different chemical substances. For example, a first oscillator 318 may have a first olfactory receptor protein layer 316 selected to measure a first chemical substance (e.g., volatile sulfur). A second oscillator 318 may have a second olfactory receptor protein layer 316 selected to measure a second chemical substance (e.g., hydrogen sulfide). By using different oscillators tuned for different chemical substances, the oral expiratory flow sensor 306 may differentiate between different chemical substances.

The oral expiratory flow sensor 306 may include frequency circuitry 320. The frequency circuitry 320 may generate an electrical signal that indicates the vibration frequency of the oscillator 318. Thus, the frequency circuitry 320 may generate a frequency signal based on the vibration of the oscillator 318. For example, the frequency circuitry 320 may generate an electrical signal corresponding to F₂ in Equation (1). In some examples, the frequency of the oscillator 318 may be converted to the mass of a given chemical substance adhered to the oscillator 318. In some examples, the mass of the chemical substance may be expressed in parts-per-million, grams, milligrams, micrograms, etc. This conversion of frequency to mass of the measured oral expiratory flow contents may occur in the oral expiratory flow sensor 306 or at a device (e.g., processor) external to the oral expiratory flow sensor 306.

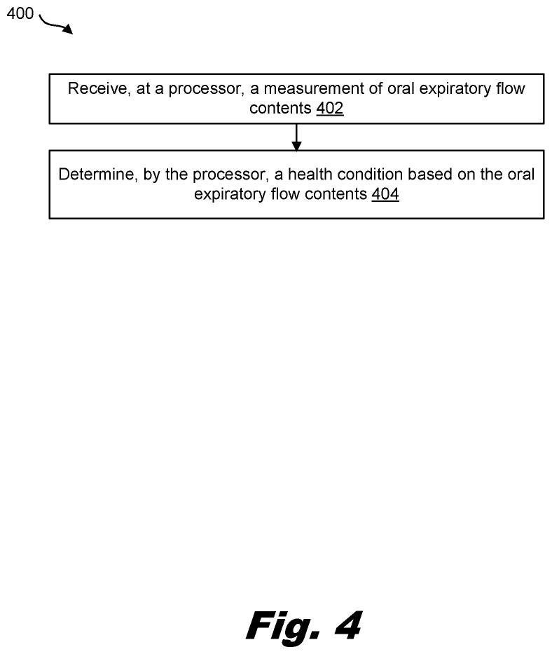

FIG. 4 is a flow diagram illustrating a method 400 for determining a health condition, according to an example. In some examples, the method 400 may be performed by a processor, such as the processor 108 of FIG. 1.

At 402, a measurement of oral expiratory flow contents may be received at the processor. For example, an oral expiratory flow sensor may measure the mass of a chemical substance. The frequency of an oscillator in the oral expiratory flow sensor may change based on the mass of the chemical substance adhered to the oscillator. The frequency of the oscillator may be used to obtain the mass of the adhered chemical substance, as illustrated by Equation (1).

At 404, a health condition may be determined, by the processor, based on the oral expiratory flow contents. In an example, determining the health condition may include detecting a bad breath or a mouth condition. For example, the processor may determine that a volatile sulfur measurement in the oral expiratory flow is greater than a first threshold (e.g., 5 ppm). The processor may also determine that a hydrogen sulfide measurement in the oral expiratory flow is greater than a second threshold (e.g., 5 ppm). The processor may determine that the health condition is bad breath or a mouth condition in response to the volatile sulfur and hydrogen sulfide measurements.

In an example, determining the health condition may include detecting a diabetes condition. For example, the processor may determine that an acetone measurement in the oral expiratory flow is greater than a first threshold (e.g., 5 ppm) and less than a second threshold (e.g., 300 ppm). The processor may then determine that the health condition is a diabetes condition in response to the acetone measurement.

In an example, determining the health condition may include detecting a liver condition. For example, the processor may determine that an ammonia measurement in the oral expiratory flow is greater than a first ammonia threshold (e.g., 1 ppm). The processor may determine that an acetic acid measurement in the oral expiratory flow is greater than a second threshold (e.g., 1 ppm) and less than a third threshold (e.g., 7 ppm). The processor may determine that the health condition is a liver condition in response to the ammonia measurement and the acetic acid measurement.

In an example, determining the health condition may include detecting a kidney condition. For example, the processor may determine that an ammonia measurement in the oral expiratory flow is greater than a second ammonia threshold (e.g., 4.8 ppm). The processor may determine that the health condition is a kidney condition in response to the ammonia measurement.

FIG. 5 depicts a non-transitory machine-readable storage medium 530 for determining a health condition, according to an example. To achieve its desired functionality, an electronic device includes various hardware components. Specifically, an electronic device includes a processor and a machine-readable storage medium 530. The machine-readable storage medium 530 is communicatively coupled to the processor. The machine-readable storage medium 530 includes a number of instructions 532, 534, 536 for performing a designated function. The machine-readable storage medium 530 causes the processor to execute the designated function of the instructions 532, 534, 536. The machine-readable storage medium 530 can store data, programs, instructions, or any other machine-readable data that can be utilized to operate the electronic device 102. Machine-readable storage medium 530 can store computer readable instructions that the processor of the electronic device 102 can process or execute. The machine-readable storage medium 530 can be an electronic, magnetic, optical, or other physical storage device that contains or stores executable instructions. Machine-readable storage medium 530 may be, for example, Random-Access Memory (RAM), an Electrically Erasable Programmable Read-Only Memory (EEPROM), a storage device, an optical disc, etc. The machine-readable storage medium 530 may be a non-transitory machine-readable storage medium 530, where the term “non-transitory” does not encompass transitory propagating signals.

Referring to FIG. 5, tracheal sound recording instructions 532, when executed by the processor, may cause the processor to receive a tracheal sound recording from a microphone device. Oral expiratory flow content instructions 534, when executed by the processor, may cause the processor to receive a measurement of oral expiratory flow contents from an oral expiratory flow sensor. Lung condition determination instructions 536 when executed by the processor, may cause the processor to determine a lung condition based on the tracheal sound recording and the oral expiratory flow contents.

In some examples, the tracheal sound recording instructions 532 may cause the processor to filter the tracheal sound recording to remove background noise. In some examples, the processor may perform adaptive thresholding to remove background noise from the tracheal sound recording.

In some examples, the instructions to determine the lung condition may include instructions executable by the processor to determine that a trimethylamine measurement in the oral expiratory flow is greater than a threshold (e.g., 5 ppm). The instructions are also executable by the processor to determine that the tracheal sound recording matches a sound signature for tracheitis. In some examples, the sound signature for tracheitis may include a wheezing sound signature, a crackle sound signature, or a stridor sound signature, as described above.

In some examples, the processor may obtain a waveform of the tracheal sound recording for two consecutive times (e.g., T1 and T2). The two consecutive times may form an area of the sound recording. The area of the sound recording may be selected based on the sound signature for a tracheitis condition. For example, wheezing may have a sound signature with a 100 ms duration, crackles may have a sound signature with a duration of 20 ms, and stridor may have a sound signature with a duration of 250 ms. Thus, in this example, the processor may obtain wave forms of 100 ms, 20 ms, and 250 ms, corresponding to each monitored lung condition.

The processor may transform the waveform of the tracheal sound recording to the frequency domain. For example, the processor may perform a fast Fourier transform (FFT) on the captured waveforms to convert the waveform to the frequency domain.

The processor may check the frequency of the waveform samples to determine whether the tracheal sound recording matches a sound signature for tracheitis. For example, for the wheezing sample (i.e., of 100 ms duration), the processor may determine whether the frequency of the wheezing sample in the frequency domain is within a frequency range for wheezing (e.g., 80 Hz<frequency<120 Hz). For the crackles sample (i.e., of 20 ms duration), the processor may determine whether the frequency of the crackles sample in the frequency domain is within a frequency range for crackles (e.g., 200 Hz<frequency<2000 Hz). For the stridor sample (i.e., of 250 ms duration), the processor may determine whether the frequency of the stridor sample in the frequency domain is within a frequency range for stridor (e.g., 350 Hz<frequency<420 Hz). If any of these conditions are met, then the processor may have detected the given lung condition. In some examples, the processor may generate an alert or other notification to indicate the presence of the lung condition.

What is claimed is:

1. An electronic device, comprising:
a microphone device to record a tracheal sound;
an oral expiratory flow sensor to measure oral expiratory flow contents; and
a processor to determine a health condition based on the tracheal sound and the oral expiratory flow contents.

2. The electronic device of claim 1, wherein the microphone device comprises:
an array of microphones; and
a support structure to position the array of microphones in proximity to a tracheal region of a human user.

3. The electronic device of claim 1, wherein the oral expiratory flow sensor to vibrate based on adhesion of the oral expiratory flow contents on the oral expiratory flow sensor.

4. The electronic device of claim 3, wherein the oral expiratory flow sensor comprises:
a filter to filter an oral expiratory flow;
an olfactory receptor protein layer to receive filtered oral expiratory flow contents;
an oscillator to vibrate with a frequency based on adhesion of the oral expiratory flow contents on the olfactory receptor protein layer; and
circuitry to generate a frequency signal based on the vibration of the oscillator.

5. The electronic device of claim 4, wherein the frequency of the oscillator changes based on changes in the oral expiratory flow contents.

6. The electronic device of claim 4, wherein the frequency of the oscillator indicates that a type of chemical substance is present in the oral expiratory flow.

7. The electronic device of claim 1, wherein the microphone device and the oral expiratory flow sensor are coupled to a headset.

8. A method, comprising:
receiving, at a processor, a measurement of oral expiratory flow contents; and
determining, by the processor, a health condition based on the oral expiratory flow contents.

9. The method of claim 8, determining the health condition comprises:
determining that a volatile sulfur measurement in the oral expiratory flow is greater than a first threshold;
determining that a hydrogen sulfide measurement in the oral expiratory flow is greater than a second threshold; and
determining that the health condition is bad breath or a mouth condition in response to the volatile sulfur and hydrogen sulfide measurements.

10. The method of claim 8, determining the health condition comprises:
determining that an acetone measurement in the oral expiratory flow is greater than a first threshold and less than a second threshold; and
determining that the health condition is a diabetes condition in response to the acetone measurement.

11. The method of claim 8, determining the health condition comprises:
determining that an ammonia measurement in the oral expiratory flow is greater than a first ammonia threshold;
determining that an acetic acid measurement in the oral expiratory flow is greater than a second threshold and less than a third threshold; and
determining that the health condition is a liver condition in response to the ammonia measurement and the acetic acid measurement.

12. The method of claim 8, determining the health condition comprises:
determining that an ammonia measurement in the oral expiratory flow is greater than a second ammonia threshold; and
determining that the health condition is a kidney condition in response to the ammonia measurement.

13. A non-transitory computer-readable storage medium comprising instructions executable by a processor to:
receive a tracheal sound recording from a microphone device;
receive a measurement of oral expiratory flow contents from an oral expiratory flow sensor; and
determine a lung condition based on the tracheal sound recording and the oral expiratory flow contents.

14. The non-transitory computer-readable storage medium of claim 13, wherein the instructions to determine the lung condition comprise instructions executable by the processor to:
determine that a trimethylamine measurement in the oral expiratory flow is greater than a threshold; and
determine that the tracheal sound recording matches a sound signature for tracheitis.

15. The non-transitory computer-readable storage medium of claim 14, wherein the sound signature for tracheitis comprises a wheezing sound signature, a crackle sound signature, or a stridor sound signature.
